(19) United States
(12) Patent Application Publication
(10) Pub. No.: US 2005/0113577 A1
(43) Pub. Date: May 26, 2005

(54) Solid forms of slats with tyrosine kinase activity

(76) Inventors: Shyam B Karki, Lansdale, PA (US); Joseph Payack, Somerset, NJ (US); Varaporn Treemaneekarn, Lansdale, PA (US); Yaling Wang, Westfield, NJ (US); Yuichi Sato, Menuma (JP)

Correspondence Address: MERCK AND CO., INC, P O BOX 2000, RAHWAY, NJ, 07065-0907, US

(21) Appl. No.: 10/506,710

(22) PCT Filed: Apr. 11, 2003
(86) PCT No.: PCT/US03/11022

Related U.S. Application Data
(60) Provisional application No. 60/372,782, filed on Apr. 16, 2002.

(51) Int. Cl. C07D 43/14; A61K 31/496

(52) U.S. Cl. 544/363; 514/253.07

(57) ABSTRACT

The present invention relates to solid forms of the hydrochloride salt 3-[5-(4-methanesulfonyl-piperazin-1-yl-methyl)-1H-indol-2-yl]-1H-quinolin-2-one which inhibit, regulate and/or modulate tyrosine kinase signal transduction, compositions which contain these compounds, and methods of using them to treat tyrosine kinase-dependent diseases and conditions, such as angio-genesis, cancer, tumor growth, atherosclerosis, age related macular degeneration, diabetic retinopathy, inflammatory diseases, and the like in mammals.

### BACKGROUND OF THE INVENTION

The present invention relates to solid forms of the hydrochloride salt of of 3-[5-(4-methanesulfonyl-piperazin-1-ylmethyl)-1H-indol-2-yl]-1H-quinolin-2-one, which inhibits tyrosine kinase signal transduction, compositions which contain these polymorphic forms, and methods of using them to treat tyrosine kinase-dependent diseases and conditions, such as angiogenesis, cancer, tumor growth, atherosclerosis, age related macular degeneration, diabetic retinopathy, inflammatory diseases, and the like in mammals.

Tyrosine kinases are a class of enzymes that catalyze the transfer of the terminal phosphate of adenosine triphosphate to tyrosine residues in protein substrates. Tyrosine kinases play critical roles in signal transduction for a number of cell functions via substrate phosphorylation and have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

Both receptor-type and non-receptor type tyrosine kinases are implicated in cellular signaling pathways leading to numerous pathogenic conditions, including cancer, psoriasis and hyperimmune responses.

Several receptor-type tyrosine kinases, and the growth factors that bind thereto, play a role in angiogenesis, although some may promote angiogenesis indirectly (Mustonen and Alitalo, J. Cell Biol. 129:895-898, 1995). One such receptor-type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Fit-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

Although 3-[5-(4-methanesulfonyl-piperazin-1-ylmethyl)-1H-indol-2-yl]-1H-quinolin-2-one has been previously reported to be useful as tyrosine kinase inhibitors (see WO 01/29025; published 26 Apr. 2001), a need still exists for forms of the compounds that can be readily administered to patients, especially orally active, soluble forms of this compounds that have thermal stability upon storage. Accordingly, the identification of solid forms of salts of the compound which specifically inhibit, regulate and/or modulate the signal transduction of tyrosine kinases is desirable and is an object of this invention.

### SUMMARY OF THE INVENTION

The present invention relates to solid forms of a hydrochloride salt of of 3-[5-(4-methanesulfonyl-piperazin-1-ylmethyl) 1H-indol-2-yl]-1H-quinolin-2-one (Compound 1) which is capable of inhibiting, modulating and/or regulating signal transduction of both receptor-type and non-receptor type tyrosine kinases.

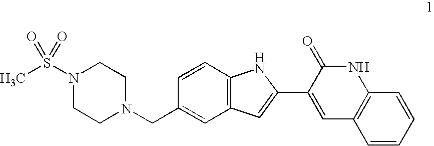

### DESCRIPTION OF THE FIGURES

FIG. 1: X-ray powder diffraction pattern of crystalline Form A of the hydrochloride salt of 3-[5-(4-methanesulfonyl-piperazin-1-ylmethyl)-1H-indol-2-yl]-1H-quinolin-2-one.

FIG. 2: X-ray powder diffraction pattern of polymorphic Form B of the hydrochloride salt of 3-[5-(4-methanesulfonyl-piperazin-1-ylmethyl)-1H-indol-2-yl]-1H-quinolin-2-one.

FIG. 3: X-ray powder diffraction pattern of crystalline Form C of the hydrochloride salt of 3-[5-(4-methanesulfonyl-piperazin-1-ylmethyl)-1H-indol-2-yl]-1H-quinolin-2-one.

FIG. 4: X-ray powder diffraction pattern of crystalline Form D of the hydrochloride salt of 3-[5-(4-methanesulfonyl-piperazin-1-ylmethyl)-1H-indol-2-yl]-1H-quinolin-2-one.

FIG. 5: X-ray powder diffraction pattern of polymorphic Form E of the hydrochloride salt of 3-[5-(4-methanesulfonyl-piperazin-1-ylmethyl)-1H-indol-2-yl]-1H-quinolin-2-one.

### DETAILED DESCRIPTION OF THE INVENTION

3-[5-(4-Methanesulfonyl-piperazin-1-ylmethyl)-1H-indol-2-yl]-1H-quinolin-2-one (Compound 1-11) is an inhibitor of tyrosine kinase signal transduction and in particularly inhibits the kinase KDR. The basic piperazine nitrogen of Compound 1-11 readily forms salts upon treatment with various acids. Such salts include, but are not limited to, mesylate, tartrate, hydrochloride, citrate, acetate, hydrobromide, maleate, sulfate and besylate. Studies on the hydrochloride salt of Compound 1-11 have revealed five distinctly different solid forms, Forms A, B, C, D and E.

It has been determined that Forms A, B and E of the hydrochloride salt of Compound 1 are anhydrous forms, while Forms C and D are hydrate forms. It has further been determined that Forms A, C and D of the hydrochloride salt of Compound 1 are crystalline forms, while Forms B and E are partially amorphous in content. Such partially crystalline, partially amorphous forms of a solid may be termed a polymorphic form. The term “polymorphic form” may also be used to describe a variety of crystalline, polymorphic and amorphic forms of a compound.

An embodiment of the invention is illustrated by Form A of the hydrochloride salt of 3-[5-(4-methanesulfonyl-piperazin-1-ylmethyl)-1H-indol-2-yl]-1H-quinolin-2-one. Form A is a crystalline form characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23 and further characterized by a melting endotherm of 295.29° C. at a rate of 10° C. per minute.

Another embodiment of the invention is illustrated by Form B of the hydrochloride salt of 3-[5-(4-methanesulfonyl-piperazin-1-ylmethyl)-1H-indol-2-yl]-1H-quinolin-2-one. Form B is a polymorphic form characterized by an X-ray powder diffraction pattern having diffraction angles of 6.76, 8.12, 10.21, 12.11, 12.88, 13.77, 14.65, 15.01, 15.23, 16.09, 16.36, 16.95, 17.28, 17.65, 18.31, 19.06, 19.66, 20.84, 21.47, 22.21, 23.07, 24.05, 24.32, 25.19, 25.58, 26.00, 26.96, 28.22 and 28.84 and further characterized by a melting endotherm of 284.90° C. at a rate of 10° C. per minute.

A further embodiment is Form D of the hydrochloride salt of 3-[5-(4-methanesulfonyl-piperazin-1-ylmethyl)-1H-indol-2-yl]-1H-quinolin-2-one. Form D is a crystalline form characterized by an X-ray powder diffraction pattern having diffraction angles of: 5.19, 9.54, 10.32, 12.99, 14.79, 15.14, 16.50, 17.10, 17.47, 18.28, 19.12, 19.50, 20.70, 21.00, 21.56, 22.27, 23.24, 24.42, 25.35, 26.06, 26.99, 28.28 and 31.87 and further characterized by a melting endotherm of 273.8° C. at a rate of 10° C. per minute.

Another embodiment is Form E of the hydrochloride salt of 3-[5-(4-methanesulfonyl-piperazin-1-ylmethyl)-1H-indol-2-yl]-1H-quinolin-2-one. Form E is a polymorphic form characterized by an X-ray powder diffraction pattern having diffraction angles of: 7.60, 9.350, 11.22, 15.12, 16.01, 16.86, 18.85, 19.46, 20.10, 21.73, 23.07, 23.70, 24.35 and 25.99 and further characterized by a melting endotherm of 292.6° C. at a rate of 10° C. per minute.

Included within the scope of this invention are methods of preparing the various forms of the hydrochloride salt of 3-(5-[4-(2-hydroxy-ethanoyl)-piperazin-1-ylmethyl]-1H-indol-2-yl)-1H-quinolin-2-one, starting with either the free base of 3-{5-[4-(2-hydroxy-ethanoyl)-piperazin-1-ylmethyl]-1H-indol-2-yl}-1H-quinolin-2-one or the hydrochloride salt of 3-{5-[4-(2-hydroxy-ethanoyl)-piperazin-1-ylmethyl]-1H-indol-2-yl}-1H-quinolin-2-one.

Form A is prepared by the treatment of the free base of 3-{5-[4-(2-hydroxy-ethanoyl)-piperazin-1-ylmethyl]-1H-indol-2-yl}-1H-quinolin-2-one in DMSO with concentrated aqueous HCl. Form A prepared in this way may be isolated by filtration and dried under anhydrous conditions, in particular under a nitrogen purge.

Form B is prepared by the treatment of the free base of 3-{5-[4-(2-hydroxy-ethanoyl)-piperazin-1-ylmethyl]-1H-indol-2-yl}-1H-quinolin-2-one in THF with concentrated aqueous HCl. Form B prepared in this way may be isolated by filtration and dried under anhydrous conditions, in particular under a nitrogen purge.

Form D is prepared by the recrystallization of Form A from 1:1 acetonitrile/water or from 1:1 acetone/water. Form D prepared in this way may be isolated by filtration.

Form E is prepared by the recrystallization of Form A from acetic acid. Form E prepared in this way may be isolated by filtration.

Also included within the scope of the claims is a pharmaceutical composition which is comprised of the polymorphic or crystalline form of the present invention and a pharmaceutically acceptable carrier. The present invention also encompasses a method of treating or preventing cancer in a mammal in need of such treatment which is comprised of administering to said mammal a therapeutically effective amount of a presently disclosed polymorphic or crystalline form. Preferred cancers for treatment are selected from cancers of the brain, genitourinary tract, lymphatic system, stomach, larynx and lung. Another set of preferred forms of cancer are histiocytic lymphoma, lung adenocarcinoma, small cell lung cancers, pancreatic cancer, gioblastomas and breast carcinoma.

Also included is a method of treating or preventing a disease in which angiogenesis is implicated, which is comprised of administering to a mammal in need of such treatment a therapeutically effective amount of a polymorphic or crystalline form of the hydrochloride salt of Compound 1-11. Such a disease in which angiogenesis is implicated is ocular diseases such as retinal vascularization, diabetic retinopathy, age-related macular degeneration, and the like.

Also included within the scope of the present invention is a method of treating or preventing inflammatory diseases which comprises administering to a mammal in need of such treatment a therapeutically effective of a polymorphic or crystalline form of the hydrochloride salt of Compound 1-11. Examples of such inflammatory diseases are rheumatoid arthritis, psoriasis, contact dermatitis, delayed hypersensitivity reactions, and the like.

Also included is a method of treating or preventing a tyrosine kinase-dependent disease or condition in a mammal which comprises administering to a mammalian patient in need of such treatment a therapeutically effective amount of a polymorphic form of the hydrochloride salt of Compound 1-11. The therapeutic amount varies according to the specific disease and is discernable to the skilled artisan without undue experimentation.

A method of treating or preventing retinal vascularization which is comprised of administering to a mammal in need of such treatment a therapeutically effective amount of a polymorphic or crystalline form of the hydrochloride salt of Compound 1 is also encompassed by the present invention. Methods of treating or preventing ocular diseases, such as diabetic retinopathy and age-related macular degeneration, are also part of the invention. Also included within the scope of the present invention is a method of treating or preventing inflammatory diseases, such as rheumatoid arthritis, psoriasis, contact dermatitis and delayed hypersensitivity reactions, as well as treatment or prevention of bone associated pathologies selected from osteosarcoma, osteoarthritis, and rickets.

The invention also contemplates the use of the instantly claimed polymorphic or crystalline forms in combination with a second compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

Preferred angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combreta-statin A4, squalamine, 6-O-chloroacetyl-carbonyl)-fumagillol, thalidomide, angiostatin, troponin-1, and an antibody to VEGF. Preferred estrogen receptor modulators are tamoxifen and raloxifene.

Also included in the scope of the claims is a method of treating cancer which comprises administering a therapeutically effective of a polymorphic or crystalline form of the hydrochloride salt of Compound 1-11 in combination with radiation therapy and/or in combination with a compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

And yet another embodiment of the invention is a method of treating cancer which comprises administering a therapeutically effective of a polymorphic or crystalline form of the hydrochloride salt of Compound 1-11 in combination with paclitaxel or trastuzumab.

Also within the scope of the invention is a method of reducing or preventing tissue damage following a cerebral ischemic event which comprises administering a therapeutically effective amount of a polymorphic or crystalline form of the hydrochloride salt of Compound 1-11.

These and other aspects of the invention will be apparent from the teachings contained herein.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like).

Compound 1-11 may exist as tautomers and both tautomeric forms are intended to be encompassed by the scope of the invention, even though only one tautomeric structure is depicted. For example, any claim to compound I below is understood to include tautomeric structure II, and vice versa, as well as mixtures thereof.

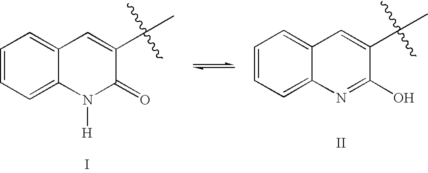

### Utility

The instant polymorphic or crystalline forms are useful as pharmaceutical agents for mammals, especially for humans, in the treatment of tyrosine kinase dependent diseases. Such diseases include the proliferation of tumor cells, the pathologic neovascularization (or angiogenesis) that supports solid tumor growth, ocular neovascularization (diabetic retinopathy, age-related macular degeneration, and the like) and inflammation (psoriasis, rheumatoid arthritis, and the like). Based on pharmacokinetic studies in animals, the presently claimed salts have an unexpectedly superior oral activity profile compared to the corresponding free base and are therefore particularly suited for oral administration. They may, however, be administered via other routes as described herein.

The polymorphic or crystalline forms of the instant invention may be administered to patients for use in the treatment of cancer. The instant polymorphic forms inhibit tumor angiogenesis, thereby affecting the growth of tumors (J. Rak et al. Cancer Research, 55:4575-4580, 1995). The anti-angiogenesis properties of the instant salts are also useful in the treatment of certain forms of blindness related to retinal vascularization.

The disclosed polymorphic or crystalline forms are also useful in the treatment of certain bone-related pathologies, such as osteosarcoma, osteoarthritis, and rickets, also known as oncogenic osteomalacia. (Hasegawa et al., Skeletal Radiol., 28, pp. 41-45, 1999; Gerber et al., Nature Medicine, Vol. 5, No. 6, pp. 623-628, June 1999). And since VEGF directly promotes osteoclastic bone resorption through KDR/Flk-1 expressed in mature osteoclasts (FEBS Let. 473:161-164 (2000); Endocrinology, 141:1667 (2000)), the instant salts are also useful to treat and prevent conditions related to bone resorption, such as osteoporosis and Paget's disease.

The claimed polymorphic or crystalline forms can also be used to reduce or prevent tissue damage which occurs after cerebral ischemic events, such as stroke, by reducing cerebral edema, tissue damage, and reperfusion injury following ischemia. (Drug News Perspect 11:265-270 (1998); J. Clin. Invest. 104:1613-1620 (1999).)

The polymorphic or crystalline forms of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice.

For oral use of a polymorphic or crystalline form according to this invention, the compound may be administered, for example, in the form of tablets or capsules, or as an aqueous solution or suspension. In the case of tablets for oral use, carriers which are commonly used include lactose and cornstarch, and lubricating agents, such as magnesium stearate, are commonly added. For oral administration in capsule form, useful diluents include lactose and dried cornstarch. When aqueous suspensions are required for oral use, the active ingredient is combined with emulsifying and suspending agents. If desired, certain sweetening and/or flavoring agents may be added.

The polymorphic or crystalline forms of the instant invention may also be co-administered with other well known therapeutic agents that are selected for their particular usefulness against the condition that is being treated. For example, in the case of bone-related disorders, combinations that would be useful include those with antiresorptive bisphosphonates, such as alendronate and risedronate; integrin blockers (defined further below), such as αᵥβ₃ antagonists; conjugated estrogens used in hormone replacement therapy, such as PREMPRO®), PREMARIN® and ENDOMETRION®); selective estrogen receptor modulators (SERMs), such as raloxifene, droloxifene, CP-336,156 (Pfizer) and lasofoxifene; cathespin K inhibitors; and ATP proton pump inhibitors.

The instant polymorphic or crystalline forms are also useful in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds which interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenylydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide, and N-4-carboxyphenyl retinamide.

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumnitepa, lobaplatin, satraplatin, profiromycin, cisplatin, irofulven, dexifosfamide, cis-aminedichloro(2-methylyridine) platinum, benzylguanine, glufosfamide, GPX100, (trans, trans, trans)-bis-mu-(hexane-21,6-diamine)-mu-[diaamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deanmino-3-aziridinyl-4-methylsulphonyl-daunorubicin.

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) enzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H, 12H-benzo[de]pyrano[3′,4′:b,7]indolizino[1,2b]quinoline-10,13(9H, 15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl) urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®); see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

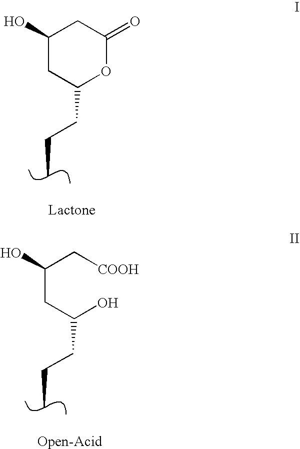

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylaamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)uinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl) 4-[1-(4-cyanobenzyl)-5-imidazolylmethyl]-5-[2-(ethanesulfonyl)methyl)-2-piperazinone, 5(S)-n-Butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-(5-[4-Hydroxymethyl-4-(4-chloropyridin-2-ylmethyl)-piperidine-1-ylmethyl]-2-methylimidazol-1-ylmethyl) benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol-4-ylmethyl) benzonitrile, 4-{3-[4-(2-Oxo-2H-[1,2′]bipyridin-5′-ylmethyl]-3H-imidazol-4-ylmethyl}benzonitrile, 4-[3-(2-Oxo-1-phenyl-1,2-dihydropyridin-4-ylmethyl)-3H-imidazol-4-ylmethyl}benzonitrile, 18,19-dihydro-19-oxo-5H, 17H-6,10:12,16-dimetheno-1H-imidazo[4,3-c][1,11,4]dioxaazacyclononadecine-9-carbonitrile, (±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (O)-19,20-Dihydro-3-methyl-19-oxo-5H-18,21-ethano-12,14-etheno-6,10-metheno-22H-benzo [d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

Examples of HIV protease inhibitors include amprenavir, abacavir, CGP-73547, CGP-61755, DMP450, indinavir, nelfinavir, tipranavir, ritonavir, saquinavir, ABT-378, AG 1776, and BMS-232,632. Examples of reverse transcriptase inhibitors include delaviridine, efavirenz, GS-840, HB Y097, lamivudine, nevirapine, AZT, 3TC, ddC, and ddI.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclo-oxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), carboxyamidotriazole, combretastatin A4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993)).

Other examples of angiogenesis inhibitors include, but are not limited to, endostation, ukrain, ranpimase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αvβ₆, αᵥβ₃₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBXI382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

The instantly claimed polymorphic or crystalline forms are also useful, alone or in combination with platelet fibrinogen receptor (GP IIb/Ia) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, Platelets 10, 285-292, 1999). Therefore, the present compounds can serve to inhibit metastasis, alone or in combination with GP IIb/IIIa) antagonists. Examples of other fibrinogen receptor antagonists include abciximab, eptifibatide, sibrafiban, lamifiban, lotrafiban, cromofiban, and CT50352.

If formulated as a fixed dose, such combination products employ the polymorphic forms of this invention within the dosage range described below and the other pharmaceutically active agent(s) within its approved dosage range. Polymorphic or crystalline forms compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the polymorphic form compound into the system of the animal in need of treatment. When a polymorphic form of the invention is provided in combination with one or more other active agents (e.g., a cytotoxic agent, etc.), “administration” and its variants are each understood to include concurrent and sequential introduction of the polymorphic form compound and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of the polymorphic or crystalline form compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The present invention also encompasses a pharmaceutical composition useful in the treatment of cancer, comprising the administration of a therapeutically effective amount of the salts of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of a polymorphic or crystalline form of the hydrochloride salt of 3-[5-(4-methanesulfonyl-piperazin-1-ylmethyl)-1H-indol-2-yl]-1H-quinolin-2-one is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

### Assays

The compounds of the instant invention described in the Examples were tested by the assays described below and were found to have kinase inhibitory activity. Other assays are known in the literature and could be readily performed by those of skill in the art (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

I. VEGF Receptor Kinase Assay

VEGF receptor kinase activity is measured by incorporation of radio-labeled phosphate into polyglutarnic acid, tyrosine, 4:1 (pEY) substrate. The phosphorylated pEY product is trapped onto a filter membrane and the incorporation of radio-labeled phosphate quantified by scintillation counting.

### Materials

VEGF Receptor Kinase

The intracellular tyrosine kinase domains of human KDR (Tennan, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

The other materials used and their compositions were as follows:
Lysis buffer: 50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).
Wash buffer: 50 mM Tris pH 7.4, 0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.
Dialysis buffer: 50 mM Tris pH 7.4, 0.5 M NaCl, 5 mM DTT, 1 mM EDTA, 0.05% triton X-100, 50% glycerol, 10 mg/mL of each leupeptin, pepstatin and aprotinin and 1 mM phenylmethylsulfonyl fluoride.
10 X reaction buffer: 200 mM Tris, pH 7.4, 1.0 M NaCl, 50 mM MnCl₂, 10 mM DTT and 5 mg/mL bovine serum albumin (Sigma).
Enzyme dilution buffer: 50 mM Tris, pH 7.4, 0.1 M NaCl, 1 mM DTT, 10% glycerol, 100 mg/mL BSA.
10 X Substrate: 750 μg/mL poly (glutamic acid, tyrosine; 4:1) (Sigma).
Stop solution: 30% trichloroacetic acid, 0.2 M sodium pyrophosphate (both Fisher).
Wash solution: 15% trichloroacetic acid, 0.2 M sodium pyrophosphate.
Filter plates: Millipore #MAFC NOB, GF/C glass fiber 96 well plate.

### Method

A. Protein purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

B. VEGF Receptor Kinase Assay

1. Add 5 μl of inhibitor or control to the assay in 50% DMSO.
2. Add 35 g of reaction mix containing 5 ill of 10× reaction buffer, 5 μl 25 mM ATP/10 μCi [³³P]ATP (Amersham), and 5 μl 10× substrate.
3. Start the reaction by the addition of 10 μL of KDR (25 nM) in enzyme dilution buffer.
4. Mix and incubate at room temperature for 15 minutes.
5. Stop by the addition of 50 μl stop solution.
6. Incubate for 15 minutes at 4° C.
7. Transfer a 90 μl aliquot to filter plate.
8. Aspirate and wash 3 times with wash solution.
9. Add 30 μl of scintillation cocktail, seal plate and count in a Wallac Microbeta scintillation counter.
   II. Human Umbilical Vein Endothelial Cell Mitogenesis Assay

Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

### Materials

HUVECs: HUVECs frozen as primary culture isolates are obtained from Clonetics Corp. Cells are maintained in Endothelial Growth Medium (EGM; Clonetics) and are used for mitogenic assays described in passages 1-5 below.
Culture Plates: NUNCLON 96-well polystyrene tissue culture plates (NUNC #167008).
Assay Medium: Dulbecco's modification of Eagle's medium containing 1 mg/mL glucose (low-glucose DIMM; Mediatech) plus 10% (v/v) fetal bovine serum (Clonetics).
Test Compounds: Working stocks of test compounds are diluted serially in 100% dimethylsulfoxide (DMSO) to 400-fold greater than their desired final concentrations. Final dilutions to IX concentration are made directly into Assay Medium immediately prior to addition to cells.
10× Growth Factors: Solutions of human VEGF165 (500 ng/mL; R&D Systems) and bFGF (10 ng/mL; R&D Systems) are prepared in Assay Medium.
10×[³H]Thymidine: [Methyl-3H]thymidine (20 Ci/mmol; Dupont-NEN) is diluted to 80 μCi/mL in low-glucose DMEM.
Cell Wash Medium: Hank's balanced salt solution (Mediatech) containing 1 mg/mL bovine serum albumin (Boehringer-Mannheim).
Cell Lysis Solution: 1 N NaOH, 2% (w/v) Na₂CO₃.

### Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μl/well of either Assay Medium, 10× VEGF solution or 10× bFGF solution. Cells are then incubated at 37° C. and 5% CO₂.

4. After 24 hours in the presence of growth factors, 10× [³H]thymidine (10 μl/well) is added.

5. Three days after addition of [³H]thymidine, medium is removed by aspiration, and cells are washed twice with Cell Wash Medium (400 μl/well followed by 200 μl/well). The washed, adherent cells are then solubilized by addition of Cell Lysis Solution (100 μl/well) and warming to 37° C. for 30 minutes. Cell lysates are transferred to 7-mL glass scintillation vials containing 150 mL of water. Scintillation cocktail (5 mL/vial) is added, and cell-associated radioactivity is determined by liquid scintillation spectroscopy.

Based upon the foregoing assays the compounds of the present invention are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC₅₀ values between 0.01-5.0 μM. These compounds may also show selectivity over related tyrosine kinases (e.g., FGFR1 and the Src family; for relationship between Src kinases and VEGFR kinases, see Eliceiri et al., Molecular Cell, Vol. 4, pp. 915-924, December 1999).

III. Flt-1 Kinase Assay

Flt-1 was expressed as a GST fusion to the Flt-1 kinase domain and was expressed in baculovirus/insect cells. The following protocol was employed to assay compounds for Flt-1 kinase inhibitory activity:
1. Inhibitors were diluted to account for the final dilution in the assay, 1:20.
2. The appropriate amount of reaction mix was prepared at room temperature:
   10× Buffer (20 mM Tris pH 7.4/0.1 M NaCl/1 mM DTT final)
   0.1M MnCl₂ (5 mM final)
   pEY substrate (75 μg/mL)
   ATP/[³³P]ATP (2.5 t/l μCi final)
   BSA (500 μg/mL final).
3. 5 μL of the diluted inhibitor was added to the reaction mix. (Final volume of 5 μL in 50% DMSO). To the positive control wells, blank DMSO (50%) was added.
4. 35 μL of the reaction mix was added to each well of a 96 well plate.
5. Enzyme was diluted into enzyme dilution buffer (kept at 4° C.).
6. 10 μL of the diluted enzyme was added to each well and mix (5 nM final). To the negative control wells, 10 μL 0.5 M EDTA was added per well instead (final 100 mM).
7. Incubation was then carried out at room temperature for 30 minutes.
8. Stopped by the addition of an equal volume (50 μL) of 30% TCA/0.1M Na pyrophosphate.
9. Incubation was then carried out for 15 minutes to allow precipitation.
10. Transferred to Millipore filter plate.
11. Washed 3× with 15% TCA/0.1M Na pyrophosphate (125 FL per wash).
12. Allowed to dry under vacuum for 2-3 minutes.
13. Dried in hood for 20 minutes.
14. Assembled Wallac Millipore adapter and added 50 μL of scintillant to each well and counted.
   IV. Flt-3 Kinase Assay

Flt-3 was expressed as a GST fusion to the Flt-3 kinase domain, and was expressed in baculovirus/insect cells. The following protocol was employed to assay compounds for Flt-3 kinase inhibitory activity:
1. Dilute inhibitors (account for the final dilution into the assay, 1:20)
2. Prepare the appropriate amount of reaction mix at room temperature. 10× Buffer (20 mM Tris pH 7.4/0.1 M NaCl/1 mM DTT final)
   0.1M MnCl₂ (5 mM final)
   pEY substrate (75 μg/mL)
   ATP/[³³P]ATP (0.5 EMIL μCi final)
   BSA (500 μg/mL final)
3. Add 5 μL of the diluted inhibitor to the reaction mix. (Final volume of 5 μL in 50% DMSO). Positive control wells—add blank DMSO (50%).
4. Add 35 μL of the reaction mix to each well of a 96 well plate.
5. Dilute enzyme into enzyme dilution buffer (keep at 4° C.).
6. Add 10 μL of the diluted enzyme to each well and mix (5-10 nM final). Negative control wells—add 10 μL 0.5 M EDTA per well instead (final 100 mM)
7. Incubate at room temperature for 60 minutes.
8. Stop by the addition of an equal volume (50 μL) of 30% TCA/0.1M Na pyrophosphate.
9. Incubate for 15 minutes to allow precipitation.
10. Transfer to Millipore filter plate.
11. Wash 3× with 15% TCA/0.1M Na pyrophosphate (125 μL per wash).
12. Allow to dry under vacuum for 2-3 minutes.
13. Dry in hood for 20 minutes.
14. Assemble Wallac Millipore adapter and add 50 μL of scintillant to each well and count.

### EXAMPLES

Examples provided are intended to assist in a further understanding of the invention. Particular materials employed, species and conditions are intended to be illustrative of the invention and not limiting of the reasonable scope thereof.

The free bases used to prepare the salts of this invention may be obtained by employing the procedures described below as well as those disclosed in WO 01/29025, published 26 Apr. 2001, hereby incorporated by reference. In addition, other procedures may be used by standard manipulations of reactions that are known in the literature.

HPLC Methods Used:

Isocratic method (for solubility studies)

| | |
|---|---|
| Column: | BDS HYPESIL, C18 (250 mm × 46 mm), 5 μm |
| | particle size |
| Column Temperature: | ambient |
| Detector: | 230 nm (UV wavelength) |
| Column Temp. | ambient |
| Flow Rate: | 1.0 mL/min |
| Injection Volume: | 20 μL |
| Mobile Phase: | A) 0.1% Phosphoric Acid |
| | B) 100% Acetonitrile |
| Diluent: | 50% Acetonitrile-DI water |
| Gradient Profile: | (A/B) starts from (60/40) and stays at (60/40) |
| | for 20 minutes. |
| Run Time: | 20 minutes |

### Example 1

### Hydrochloride Salt of 3-[5-(4-Methanesulfonyl-piperazin-1-ylmethyl)-1H-indol-2-yl]-1H-quinolin-2-one (1-11)

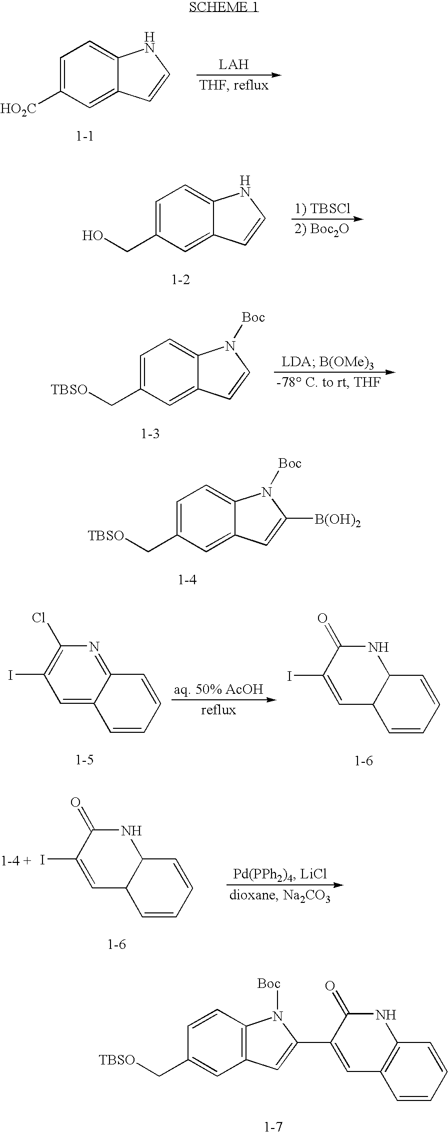

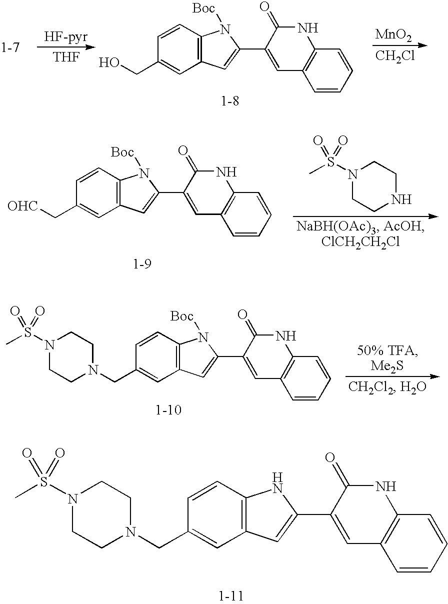

### (1H-Indol-5-yl)-methanol (1-2)

To a mechanically stirred solution of 1H-Indole-5-carboxylic acid (1-1, 20.01 g, 124 mmol) in THF (500 mL) was added at ambient temperature slowly a solution of IM-LAH in toluene (186 mL, 186 mmol, 1.5 equiv). The reaction mixture was heated at reflux for 1 hour, quenched with ice, partitioned between ethylacetate and saturated aqueous NaHCO₃. The organic layer was washed with brine, separated, dried (MgSO₄) and concentrated in vacuo. The crude product solidified upon standing under the reduced pressure. The crude solid was suspended in hexanes (200 μL) and ethyl acetate (10 mL), stirred overnight, collected by filtration and air-dried to afford the desired product as a light brown solid. ¹H NMR (400 Nz, CDCl₃) δ 8.24 (br s, 1H), 7.62 (s, 1H), 7.36 (d, 1H, J=8.4 Hz), 7.23 (d, 1H, J=8.4 Hz), 7.20 (s, 1H), 6.54 (s, 1H), 4.75 (s, 2H), 1.68 (s, 1H).

### 5-(tert-Butyl-dimethyl-silanyloxymethyl)-indole-1-carboxylic Acid tert-butyl Ester (1-3)

A stirred solution of (1H-Indol-5-yl)-methanol (1-2, 16.5 g, 112.1 mmol) in dichloromethane (300 mL) was subsequently treated at ambient temperature with diisopropylethylamine (39 mL, 224.2 mmol, 2 equiv), tert-butyldimethylsilyl chloride (18.6 g, 123.3 mmol, 1.1 equiv), and 4-(N,N-dimethylamino)pyridine (1.37 g, 11.2 mmol, 0.1 equiv). The reaction mixture was stirred at room temperature for 30 minutes, concentrated in vacuo, partitioned between ethyl acetate and 0.5N—HCl. The organic layer was washed with brine, separated, dried (MgSO₄), concentrated in vacuo to give the crude silylether as a light brown solid. The crude product and di-tert-butyl dicarbonate (26.9, 123.3 mmol) were dissolved in dichrolomethane (300 mL) and stirred at ambient temperature in the presence of 4-(NN-dimethylamino) pyridine (1.37 g, 11.2 mmol) for 2 hours. The reaction mixture was concentrated in vacuo, partitioned between ethyl acetate and 0.5N—HCl. The organic layer was washed with brine, separated, dried (MgSO₄) and concentrated in vacuo to give the crude oil. Chromatography (SiO₂, 10% ethyl acetate in hexanes) afforded 5-(tert-Butyl-dimethyl-silanyloxymethyl)-indole-1-carboxylic acid tert-butyl ester (1-3) as a white solid; ¹H NMR (400 MHz, CDCl₃) δ7.97 (d, 1H, J=8.0 Hz), 7.47 (d, 1H, J=3.2 Hz), 7.41 (s, 1H), 7.15 (d, 1H, J=7.7 Hz), 6.44 (d, 1H, J=3.6 Hz), 4.72 (s, 2H), 1.56 (s, 9H), 0.84 (s, 9H), 0.00 (s, 6H).

### 5-(tert-Butyl-dimethyl-silanyloxymethyl)-indole-1-tert-butyloxycarbonylindole-2-boronic Acid (1-4)

To a stirred solution of 5-(tert-Butyl-dimethyl-silanyloxymethyl)-indole-1-carboxylic acid tert-butyl ester (1-3, 38.6 g, 106.7 mmol) in tetrahydrofuran (400 mL) was slowly added at −78° C. a solution of lithiun diisopropylamide in tetrahydrofuran (2M, 80.1 mL, 160.1 mmol, 1.5 equiv). The reaction mixture was stirred at the same temperature for 1 hour, treated with trimethylborate, warmed up to ambient temperature, and partitioned between ethyl acetate and 0.5N—HCl. The organic layer was washed with brine, separated, dried (MgSO₄) and concentrated in vacuo to give the crude solid. Trituation of the crude product with hexanes followed by filtration and air-drying afforded the desired boronic acid (1-4) as a white powder. ¹H NMR (400 MHz, CDCl₃) δ 7.96 (d, 1H, J=6.8 Hz), 7.54 (s, 1H), 7.47 (s, 1H), 7.32 (d, 1H, J=6.8 Hz), 7.10 (s, 1H), 4.82 (s, 2H), 1.74 (s, 9H), 0.95 (s, 9H), 0.11 (s, 6H).

### 3-Iodo-1H-quinolin-2-one (1-6)

The 2-chloro-3-iodoquinoline (1-5, 30.0 g) was weighed into a 250 mL flask and suspended in of 50% aqueous acetic acid (125 mL). The mixture was heated to 100° C. and allowed to reflux for 16 hours to completion by TLC analysis of the crude reaction mixture. The mixture was allowed to cool to ambient temperature followed by dilution with 200 mL of water. The resulting suspension of the desired product was isolated by vacuum filtration follows by washing with water (50 mL). The water and traces of acetic acid were removed under vacuum for 5 hours to afford the desired quinolinone as a tan powder (1-6). ¹H NMR (500 MHz, CDCl₃) δ 12.13 (br s, 1H), 8.71 (s, 1H), 7.65 (d, 1H, J=7.5 Hz), 7.54 (m, 1H), 7.31 (d, 1H, J=8.0 Hz), 7.20 (m, 1H).

### 5-Hydroxymethyl-2-(2-oxo-1,2-dihydro-quinolin-3-yl)-indole-1-carboxylic Acid tert-butyl Ester (1-8)

A stirred mixture of the iodoquinolinone (1-6, 10 g, 36.9 mmol, 1 equiv), the boronic acid (1-4, 7.5 g, 18.45 mmol, 0.5 equiv), tetrakis (triphenyl-phosphine) palladium (1.71 g, 1.48 mmol, 0.04 equiv), and lithium chloride (4.69 g, 110.7 mmol, 3 equiv) in dioxane/2M-aqueous Na₂CO₃ was degassed and heated at 80° C. until the boronic acid is not detected by thin layer chromatography. Additional boronic acid (0.2 equiv at a time) was added to the reaction mixture until all the iodoquinolinone (1-6) was consumed completely (1.5 equivalent of the boronic acid, 1-4, in total, was required). The reaction mixture was partitioned between ethyl acetate and saturated aqueous NaHCO₃. The organic layer was washed with brine, separated, dried (MgSO₄) and concentrated in vacuo. The crude oil (1-7) was dissolved in tetrahydrofuran (100 mL), transferred to the PEG bottle, treated at 0° C. with HF-pyridine (15 mL) and stirred for 1 hour at ambient temperature. The reaction mixture was partitioned between ethyl acetate and saturated aqueous NaHCO₃. The organic layer was washed with brine, separated, dried (MgSO₄) and concentrated in vacuo. The crude solid was trituated with ethyl acetate and hexanes, collected by filtration and air-dried to afford the desired product (1-8) as a light yellow solid; ¹H NMR (500 MHz, DMSO-d₆) δ 12.1 (s, 1H), 8.07 (s, 1H), 8.03 (d, 1H, J=8.5 Hz), 7.74 (d, 1H, J=7.5 Hz), 7.55 (s, 1H), 7.52 (t, 1H, J=7.5 Hz), 7.35 (d, 1H, J=8.5 Hz), 7.30 (d, 1H, J=7.5 Hz), 7.22 (t, 1H, J=7.5 Hz), 6.77 (s, 11H), 5.21 (t, 1H, J=5.5 Hz), 4.60 (d, 2H, J=5.5 Hz), 1.35 (s, 9H).

### 5-Formyl-2-(2-oxo-1,2-dihydro-quinolin-3-yl)-indole-1-carboxylic Acid tert-butyl Ester (1-9)

The pre-activated MnO₂ (34.5 g, 15 equiv) and the alcohol (1-8, 10.32 g, 1.0 equiv) were weighed into a 1-liter flask and suspended in dry dichloromethane (500 mL). The reaction mixture was heated to 45° C. and was complete by thin layer chromatography after 1 hour. The mixture was allowed to cool to ambient temperature and the manganese oxide(s) were removed by vacuum filtration. The resulting pad of oxides on the filter were triturated with hot THF and the solvent filtered through under vacuum to remove any product from the oxides. The resulting filtrate was concentrated in vacuo to afford the crude aldehyde as a yellow solid. The solid was triturated with methanol (10 mL) and ethyl acetate (15 mL) followed by vacuum filtration to isolate the pure product. The light-yellow aldehyde was dried under vacuum (1-9). ¹H NMR (500 MHz, DMSO-d₆) δ 12.15 (s, 1H), 10.08 (s, 1H), 8.26 (d, 1H, J=1.5 Hz), 8.24 (d, 1H, J=8.5 Hz), 8.15 (s, 1H), 7.90 (dd, 1H, J=8.5, 1.5 Hz), 7.77 (d, 1H, J=7.5 Hz), 7.55 (m, 1H), 7.37 (d, 1H, J=8.5 Hz), 7.24 (m, 1H), 7.01 (s, 1H).

### 5-(4-Methanesulfonyl-piperazin-1-ylmethyl)-2-(2-oxo-1,2-dihydro-quinolin-3-yl)-indole-1-carboxylic Acid tert-butyl Ester (1-10)

To a stirred solution of the aldehyde (1-9, 2.01 g, 5.15 mmol, 1 equiv) and N-methanesulfonylpiperazine acetic acid salt (4.62 g, 20.60 mmol, 4 equiv) in dichloroethane (400 mL) was added at ambient temperature acetic acid (1.2 mL). The reaction mixture was treated with sodium triacetoxyborohydride and stirred for 3 hours. The reaction stopped at 76% of conversion and treated with MgSO₄ and additional 1 g of the hydride. After further stirring for 1 hour the reaction was complete. The reaction mixture was partitioned between ethyl acetate and saturated aqueous NaHCO₃. The organic layer was once again washed with saturated aqueous NaHCO₃, and then with brine, separated, dried with (Na₂SO₄) and concentrated in vacuo. The crude solid was dissolved in dimethylformamide and treated with the activated carbon. The filtrate solution (celite) was concentrated to syrup which was quickly trituated with methanol (100 mL). The resulting solid was collected by filtration, redissolved in dimethylformamide, concentrated to syrup, trituated with methanol (100 mL), collected by filtration and vacuum-dried to give 5-(4-Methanesulfonyl-piperazin-1-ylmethyl)-2-(2-oxo-1,2-dihydro-quinolin-3-yl)-indole-1-carboxylic acid tert-butyl ester (1-10) as a white powder; ¹H NMR (500 MHz, DMSO-d₆) δ 12.06 (s, 1H), 8.06 (s, 1H), 8.04 (d, 1H, J=8.5 Hz), 7.74 (d, 1H, J=8.0 Hz), 7.55 (s, 1H), 7.53 (dt, 1H, J=8.0, 1.5 Hz), 7.35 (d, 1H, J=8.5 Hz), 7.30 (dd, 1H, J=8.5, 1.5 Hz), 7.22 (t, 1H, J=7.5 Hz), 6.76 (s, 1H), 3.62 (s, 2H), 3.16 (m, 4H), 2.87 (s, 3H), 2.48 (m, 4H), 1.35 (s, 9H).

### 3-[5-(4-Methanesulfonyl-piperazin-1-ylmethyl)-1H-indol-2-yl]-1H-quinolin-2-one (1-11)

A mixture of 5-(4-Methanesulfonyl-piperazin-1-ylmethyl)-2-(2-oxo-1,2-dihydroquinolin-3-yl)-indole-1-carboxylic acid tert-butyl ester (1-10, 1.02 g, 1.863 mmol), dimethylsulfide (1.2 mL), water (0.6 mL) and TFA (40 mL) in dichloromethane (40 mL) was stirred for 1.5 hours. The reaction mixture was concentrated in vacuo, partitioned between ethyl acetate and saturated aqueous NaHCO₃. The organic layer was washed with brine, separated, dried (Na₂SO₄), and concentrated in vacuo. The resulting crude solid was purified by reverse-phase liquid chromatography (H₂O/CH₃CN gradient with 0.1% TFA present) to give trifluoroacetic acid salt of 1-11. All the fractions containing the desired product was partitioned between ethyl acetate and saturated aqueous NaHCO₃. The organic layer was washed with brine, separated, dried (Na₂SO₄), and concentrated in vacuo to give 3-[5-(4-Methanesulfonyl-piperazin-1-ylmethyl)-1H-indol-2-yl]-1H-quinolin-2-one (1-11) as a bright yellow solid; ¹H NMR (500 MHz, DMSO-d₆) δ 12.07 (s, 1H), 11.54 (s, 1H), 8.53 (s, 1H), 7.73 (d, 1H, J=7.5 Hz), 7.52 (t, 1H, J=7.5 Hz), 7.47-7.46 (m, 2H), 7.38 (d, 1H, J=8.5 Hz), 7.29 (br s, 11H), 7.25 (t, 1H, J=7.5 Hz), 7.08 (d, 1H, J=9.0 Hz), 3.57 (s, 214), 3.11 (m, 4H), 2.87 (s, 3H), 2.48 (m, 4H).

Solid Forms of the HCl Salt:

Form A:

The crude free base 1-11 described above (2.4 Kg, 5.48 moles) was charged into a 100 L flask, and DMSO was added (19 L). Concentrated aqueous HCL was added (500 mL), then the batch was seeded (24 g). The seedbed was aged 1 h, then EtOH (48 L) was added over 4 h (cubic addition: hour one, 4 L; hour two, 8 L; hour three, 16 L, hour four, 20 L). The mixture was aged an additional 1 h, then filtered. The solid was washed with 5 L 3:1 EtOH/DMSO, then with 5 L EtOH. The solid was then dried at 55° C. under a N₂ purge. The crystalline solid thus obtained was designated Form A.

Recrystallization of Form A from 1:4 acetic acid/diethyl ether provides Form A crystalline solid.

Microscopic Characteristics

Polarized light optical microscopy of Form A shows needle-like yellow particles of approximately 25-100 cm, which are highly birefringent under polarized light.

X-Ray Powder Diffraction (XRPD)

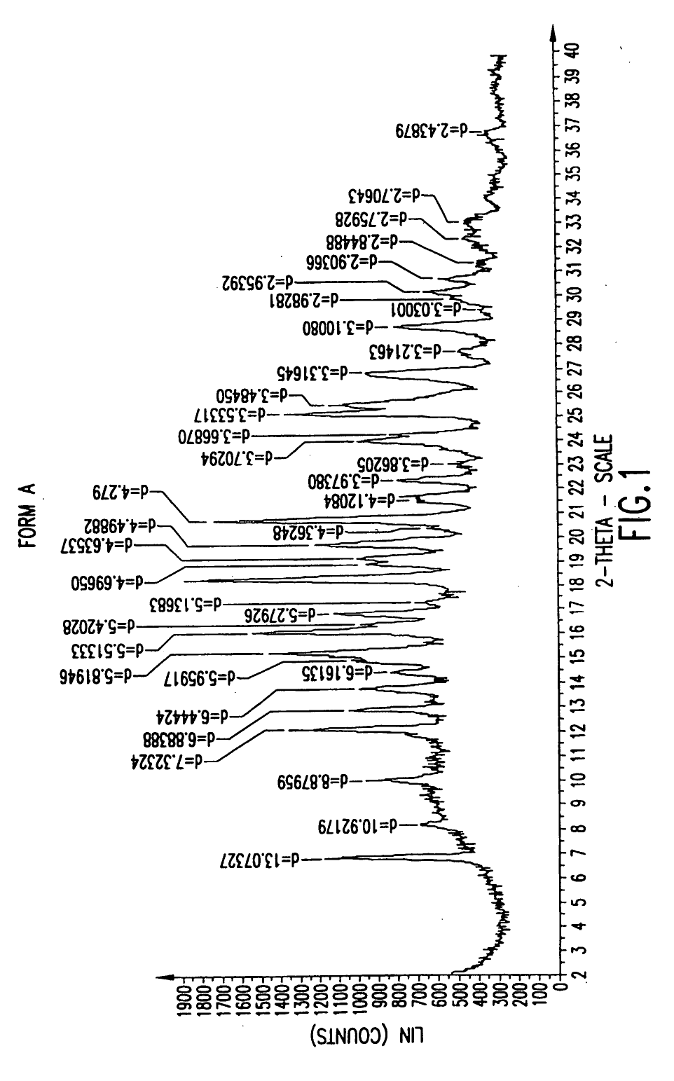

An X-ray powder diffraction pattern of Form A is indicative of a crystalline material with multiple diffraction peaks between 5° and 30° 2-theta. No change in crystal structure is observed when this form is suspended in aqueous ethanol for seven days at RT (as determined by XRPD, see FIG. 1).

Thermal Properties

DSC

Differential scanning calorimetry (DSC) of Form A from 20° C. to 350° C. at a heating rate of 10° C./min. shows a sharp endotherm at 295.3° C., which is attributed to melting. TGA and DSC traces suggest that Form A is an anhydrous polymorph and decomposes upon melting as determined by a sharp weight loss in the TGA at the melting temperature.

TGA

Thermogravimetric analysis (TGA) of Form A from 20° C. to 400° C. at a heating rate of 10° C./min. shows a weight loss of 0.54% between 20° C. and 150° C. This weight loss is attributed to adsorbed residual moisture.

Hygroscopicity

The hygroscopicity of Form A was determined at 25° C. using a step isotherm program for relative humidities from 0 to 95% RH under nitrogen flow. The Form A HCl salt is not hygroscopic at 25° C. and reversibly adsorbs approximately 1% moisture at 75% RH (relative humidity).

Solubility

The solubility of Form A HCl Salt at room temperature was determined in water and several organic solvents that can be used in pharmaceutical processing. The results are tabulated in Table I.

TABLE I
Solubility of Form A in water and various organic solvents
at room temperature (suspended for 7 days).

| Solvents | Solubility (mg/mL) |
|---|---|
| Water | 0.26 |
| Ethanol | 0.13 |
| Isopropanol | 0.057 |
| Aqueous ETOH (50%) | 4.34 |
| Aqueous IPA (50%) | 3.99 |

The solubility of Form A in various aqueous solvent systems was measured for a period of 20 hours. The results are shown below in Table II.

TABLE II
Solubility of Form A in Various Solvent Systems

| Solvent | Solubility (mg/mL) |
|---|---|
| 10% IPA | 0.47 |
| 25% IPA | 1.78 |
| 50% IPA | 4.39 |
| 75% IPA | 2.29 |
| 10% EtOH | 0.38 |
| 25% EtOH | 1.06 |
| 50% EtOH | 3.82 |
| 75% EtOH | 3.86 |
| Acetone | 0.061 |
| 50% Acetone | 10.66 |

Partition Coefficient

Partition coefficient of Form A between I-octanol and water were determined at the native pH of the HCl Salt (Table III).

TABLE III
Partition Coefficients of Form A

| pH (equilibrium) | L-021649¹ | K_{o/w} | log P (log K_{o/w}) |
|---|---|---|---|
| 3.8 (native) | 0.033 mg/mL | 0.999 | −4.34 |

¹The concentration of Form A in the aqueous phase at the start of the experiment

Form B:

The crude free base 1-11 described above was slurried in TIRF at room temperature and concentrated aqueous HCL was added slowly. The mixture was aged an hour, then filtered. The solid was washed with THF. The solid was then dried at 55° C. under a N₂ purge. The crystalline solid thus obtained was designated Form B. The microscopic characterization of Form B was as irregularly shaped crystals and partially amorphous.

Differential scanning calorimetry (DSC) of Form B from 20° C. to 350° C. at a heating rate of 10° C./min. shows a sharp endotherm at 284.9° C., which is attributed to melting. Thermogravimetric analysis (TGA) of Form B from 20° C. to 400° C. at a heating rate of 10° C./min. shows a weight loss of 0.23% between 20° C. and 150° C. TGA and DSC traces suggest that Form B is an anhydrous polymorph and decomposes upon melting as determined by a sharp weight loss in the TGA at the melting temperature.

Form C:

A stirred slurry of the crude free base 1-11 described above (1.02 g) in methanol (500 mL) was treated at the ambient temperature with aqueous HCl solution (IN, 2.30 mL). The resulting yellow solution was concentrated in vacuo. The residual solid was suspended in ethyl acetate (30 mL), filtered, washed with ethyl acetate and dried under the reduced pressure. The crystalline solid thus obtained was designated Form C. The microscopic characterization of Form C was as rod and plate shaped crystals.

Differential scanning calorimetry (DSC) of Form C from 20° C. to 350° C. at a heating rate of 10° C./min. shows a melting endotherm at 285.3° C., which is attributed to melting. Thermogravimetric analysis (TGA) of Form C from 20° C. to 400° C. at a heating rate of 10° C./min. shows a weight loss of 5% between 20° C. and 150° C. TGA and DSC traces suggest that Form C is a hydrate and decomposes upon melting as determined by a sharp weight loss in the TGA at the melting temperature.

Form D:

The Form A HCl salt described above was dissolved in a hot 1:1 acetonitrile/water mixture and the mixture filtered while hot. The filtrate solution was allowed to cool to room temperature, then slowly cooled to 5° C. The resulting crystalline solid was collected by filtration and dried under the reduced pressure. The crystalline solid thus obtained was designated Form D. The microscopic characterization of Form D was as plate shaped crystals.

Differential scanning calorimetry (DSC) of Form D from 20° C. to 350° C. at a heating rate of 10° C./min. shows a melting endotherm at 273.8° C., which is attributed to melting. Thermogravimetric analysis (TGA) of Form C from 20° C. to 400° C. at a heating rate of 10° C./min. shows a weight loss of 0.89-2.45% between 20° C. and 150° C. TGA and DSC traces suggest that Form D is a hydrate and decomposes upon melting as determined by a sharp weight loss in the TGA at the melting temperature.

Recrystallization of the Form A HCl salt from hot 1:1 acetone/water also provided Form D.

Form E:

The Form A HCl salt described above was dissolved in hot acetic acid and the mixture filtered while hot. The filtrate solution was allowed to cool to room temperature, then slowly cooled to −20° C. The resulting mixture was allowed to slowly warm to room temperature and the crystalline solid was collected by filtration and dried overnight under the reduced pressure. The crystalline solid thus obtained was designated Form E. The microscopic characterization of Form E was as rod and plate shaped crystals.

Differential scanning calorimetry (DSC) of Form E from 20° C. to 350° C. at a heating rate of 10° C./min. shows a melting endotherm at 292.6° C., which is attributed to melting. Thermogravimetric analysis (TGA) of Form E from 20° C. to 400° C. at a heatin 1 g rate of 10° C./min. shows a weight loss of 2.55% between 20° C. and 150° C. TGA and DSC traces suggest that Form E is anhydrous and decomposes upon melting as determined by a sharp weight loss in the TGA at the melting temperature.

A slurry of Form E HCl salt in water at room temperature for seven days provided Form D crystalline solid.

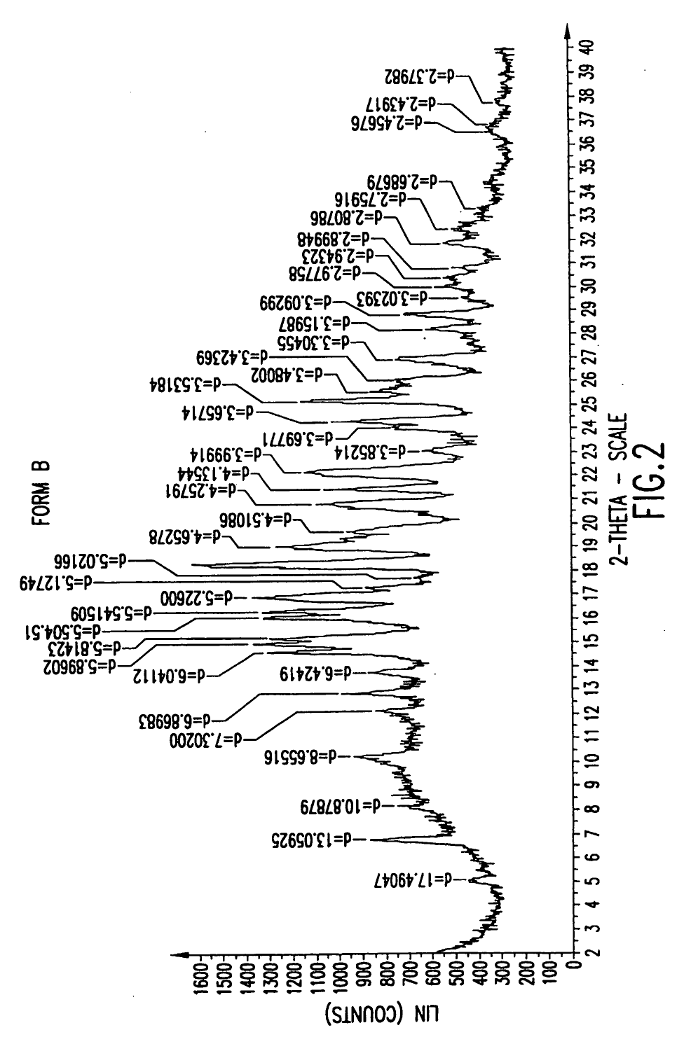

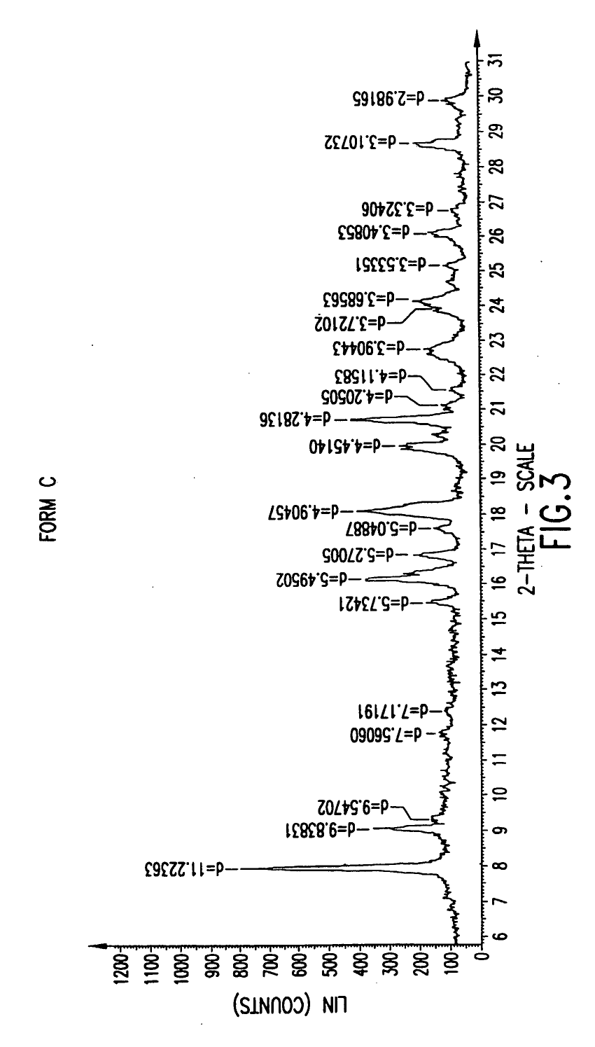

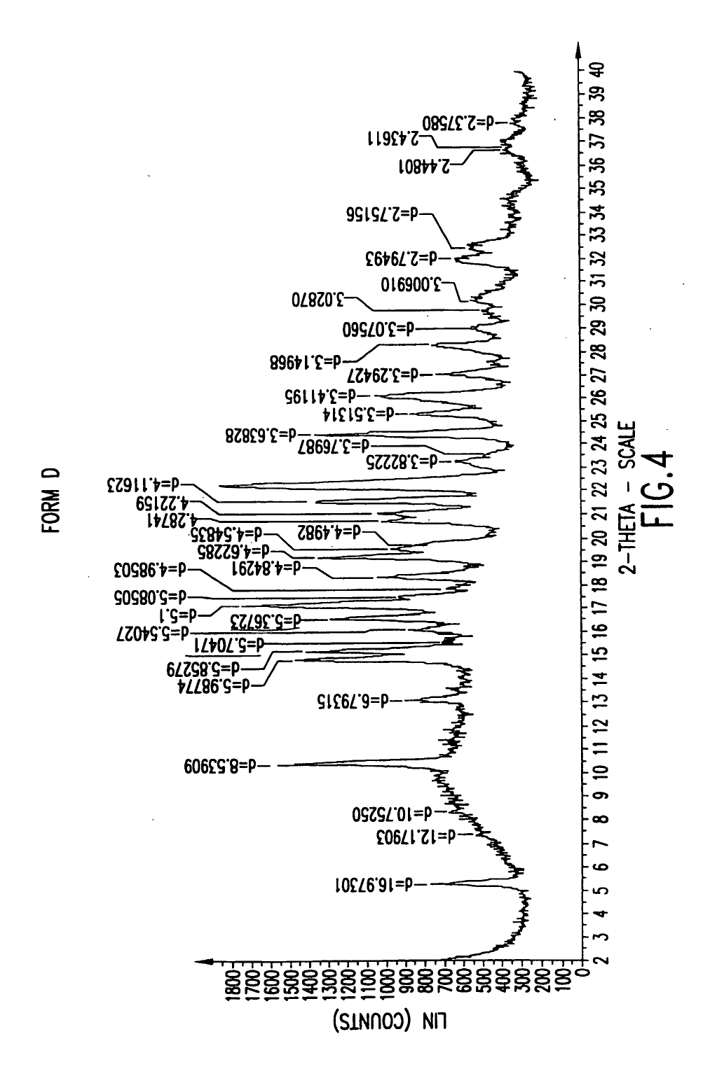

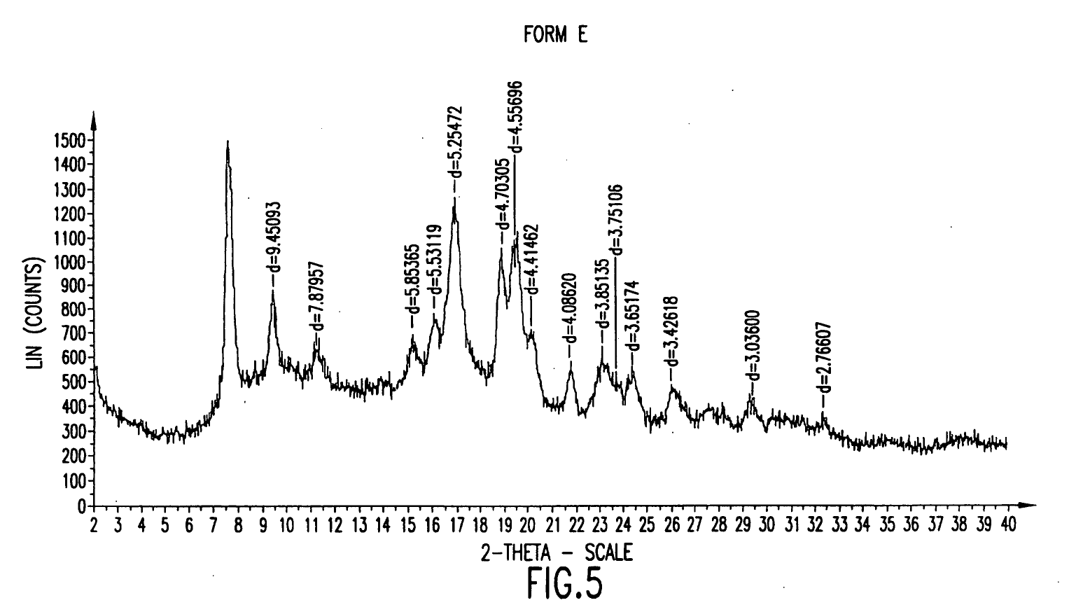

The X-ray powder diffraction patterns for Forms A-E are illustrated in FIGS. 1 to 5. The X-ray powder diffraction data for Forms A-E is summarized below in the tables below:

Form A

| d value | 2-Theta ° | % Intensity |
|---|---|---|
| =13.07 | 6.76 | 62.1 |
| =10.92 | 8.09 | 37.4 |
| =8.88 | 9.95 | 50.4 |
| =7.32 | 12.07 | 71.4 |
| =6.88 | 12.85 | 55.5 |
| =6.44 | 13.73 | 52.4 |
| =6.16 | 14.36 | 44.3 |
| =5.96 | 14.85 | 54.8 |
| =5.82 | 15.21 | 76.9 |
| =5.51 | 16.06 | 80.7 |
| =5.42 | 16.34 | 52.4 |
| =5.28 | 16.78 | 58.6 |
| =5.14 | 17.25 | 37.4 |
| =4.85 | 18.29 | 100.0 |
| =4.70 | 18.88 | 51.2 |
| =4.63 | 19.13 | 52.4 |
| =4.50 | 19.72 | 64.3 |
| =4.36 | 20.34 | 35.3 |
| =4.28 | 20.74 | 91.5 |
| =4.12 | 21.55 | 37.8 |
| =3.97 | 22.35 | 42.1 |
| =3.70 | 24.01 | 52.5 |
| =3.67 | 24.24 | 43.7 |
| =3.53 | 25.19 | 71.6 |
| =3.48 | 25.54 | 60.2 |
| =3.32 | 26.86 | 49.9 |
| =3.10 | 28.77 | 42.9 |
| =2.95 | 30.23 | 35.0 |

Form B

| d value | 2-Theta ° | % Intensity |
|---|---|---|
| =13.06 | 6.76 | 53.5 |
| =10.88 | 8.12 | 45.8 |
| =8.65 | 10.21 | 58.0 |
| =7.30 | 12.11 | 51.9 |
| =6.87 | 12.88 | 61.0 |
| =6.42 | 13.77 | 53.7 |
| =6.04 | 14.65 | 80.1 |
| =5.90 | 15.01 | 84.5 |
| =5.81 | 15.23 | 78.9 |
| =5.50 | 16.09 | 81.4 |
| =5.41 | 16.36 | 81.4 |
| =5.23 | 16.95 | 84.3 |
| =5.13 | 17.28 | 54.3 |
| =5.02 | 17.65 | 41.1 |
| =4.84 | 18.31 | 100.0 |
| =4.65 | 19.06 | 78.4 |
| =4.51 | 19.66 | 59.0 |
| =4.26 | 20.84 | 67.5 |
| =4.13 | 21.47 | 64.6 |
| =4.00 | 22.21 | 72.8 |
| =3.85 | 23.07 | 38.9 |
| =3.70 | 24.05 | 47.0 |
| =3.66 | 24.32 | 62.9 |
| =3.53 | 25.19 | 72.5 |
| =3.48 | 25.58 | 52.8 |
| =3.42 | 26.00 | 45.2 |
| =3.30 | 26.96 | 46.3 |
| =3.16 | 28.22 | 37.7 |
| =3.09 | 28.84 | 43.9 |

Form C

| d value | 2-Theta ° | % Intensity |
|---|---|---|
| =11.22 | 7.87 | 100.0 |
| =9.84 | 8.98 | 44.5 |
| =5.50 | 16.12 | 49.1 |
| =5.27 | 16.81 | 28.7 |
| =4.90 | 18.07 | 51.2 |
| =4.45 | 19.93 | 34.6 |
| =4.28 | 20.73 | 53.8 |
| =3.90 | 22.76 | 23.6 |
| =3.69 | 24.13 | 28.7 |
| =3.41 | 26.12 | 21.3 |
| =3.11 | 28.71 | 26.6 |

Form D

| d value | 2-Theta ° | % Intensity |
|---|---|---|
| =17.01 | 5.19 | 50.7 |
| =9.26 | 9.54 | 38.0 |
| =8.56 | 10.32 | 100.0 |
| =6.81 | 12.99 | 42.7 |
| =5.99 | 14.79 | 61.2 |
| =5.85 | 15.14 | 52.6 |
| =5.37 | 16.50 | 57.2 |
| =5.18 | 17.10 | 70.0 |
| =5.07 | 17.47 | 40.7 |
| =4.85 | 18.28 | 46.9 |
| =4.64 | 19.12 | 68.0 |
| =4.55 | 19.50 | 50.5 |
| =4.29 | 20.70 | 68.6 |
| =4.23 | 21.00 | 57.9 |
| =4.12 | 21.56 | 59.0 |
| =3.99 | 22.27 | 92.5 |
| =3.82 | 23.24 | 32.2 |
| =3.64 | 24.42 | 63.5 |
| =3.51 | 25.35 | 37.3 |
| =3.42 | 26.06 | 63.4 |
| =3.30 | 26.99 | 40.9 |
| =3.15 | 28.28 | 46.1 |
| =2.81 | 31.87 | 36.4 |

Form E

| d value | 2-Theta ° | % Intensity |
|---|---|---|
| =11.62 | 7.60 | 100.0 |
| =9.45 | 9.350 | 58.2 |
| =7.88 | 11.22 | 41.2 |
| =5.85 | 15.12 | 46.5 |
| =5.53 | 16.01 | 51.9 |
| =5.25 | 16.86 | 83.6 |
| =4.70 | 18.85 | 69.9 |
| =4.56 | 19.46 | 72.1 |
| =4.41 | 20.10 | 47.5 |
| =4.09 | 21.73 | 38.9 |
| =3.85 | 23.07 | 41.7 |
| =3.75 | 23.70 | 35.6 |
| =3.65 | 24.35 | 37.1 |
| =3.43 | 25.99 | 32.7 |

1. A hydrochloride salt of 3-[5-(4-methanesulfonyl-piperazin-1-ylmethyl)-1H-indol-2-yl]-1H-quinolin-2-one in a polymorphic form characterized by an X-ray powder diffraction pattern having diffraction angles of 6.76, 8.12, 10.21, 12.11, 12.88, 13.77, 14.65, 15.01, 15.23, 16.09, 16.36, 16.95, 17.28, 17.65, 18.31, 19.06, 19.66, 20.84, 21.47, 22.21, 23.07, 24.05, 24.32, 25.19, 25.58, 26.00, 26.96, 28.22 and 28.84.

2. The polymorphic form of the hydrochloride salt of 3-[5-(4-methanesulfonyl-piperazin-1-ylmethyl)-1H-indol-2-yl]-1H-quinolin-2-one according to claim 1 characterized by an X-ray powder diffraction pattern having diffraction angles of 6.76, 8.12, 10.21, 12.11, 12.88, 13.77, 14.65, 15.01, 15.23, 16.09, 16.36, 16.95, 17.28, 17.65, 18.31, 19.06, 19.66, 20.84, 21.47, 22.21, 23.07, 24.05, 24.32, 25.19, 25.58, 26.00, 26.96, 28.22 and 28.84 and a melting endotherm of 284.90° C. at a rate of 10° C. per minute.

3. A hydrochloride salt of 3-[5-(4-methanesulfonyl-piperazin-1-ylmethyl)-1H-indol-2-yl]-1H-quinolin-2-one in crystalline form characterized by an X-ray powder diffraction pattern having diffraction angles of 5.19, 9.54, 10.32, 12.99, 14.79, 15.14, 16.50, 17.10, 17.47, 18.28, 19.12, 19.50, 20.70, 21.00, 21.56, 22.27, 23.24, 24.42, 25.35, 26.06, 26.99, 28.28 and 31.87.

4. The crystalline form of the hydrochloride salt of 3-[5-(4-methanesulfonyl-piperazin-1-ylmethyl)-1H-indol-2-yl]-1H-quinolin-2-one according to claim 3 characterized by an X-ray powder diffraction pattern having diffraction angles of 5.19, 9.54, 10.32, 12.99, 14.79, 15.14, 16.50, 17.10, 17.47, 18.28, 19.12, 19.50, 20.70, 21.00, 21.56, 22.27, 23.24, 24.42, 25.35, 26.06, 26.99, 28.28 and 31.87 and a melting endotherm of 273.8° C. at a rate of 10° C. per minute.

5. A hydrochloride salt of 3-[5-(4-methanesulfonyl-piperazin-1-ylmethyl)-1H-indol-2-yl]-1H-quinolin-2-one in a polymorphic form characterized by an X-ray powder diffraction pattern having diffraction angles of 7.60, 9.350, 11.22, 15.12, 16.01, 16.86, 18.85, 19.46, 20.10, 21.73, 23.07, 23.70, 24.35 and 25.99.

6. The polymorphic form of the hydrochloride salt of 3-[5-(4-methanesulfonyl-piperazin-1-ylmethyl)-1H-indol-2-yl]-1H-quinolin-2-one according to claim 5 characterized by an X-ray powder diffraction pattern having diffraction angles of 7.60, 9.350, 11.22, 15.12, 16.01, 16.86, 18.85, 19.46, 20.10, 21.73, 23.07, 23.70, 24.35 and 25.99 and a melting endotherm of 292.6° C. at a rate of 10° C. per minute.

7. A method of preparing a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23; said method comprising the step of treating the free base of 3-[5-(4-methanesulfonyl-piperazin-1-ylmethyl)-1H-indol-2-yl]-1H-quinolin-2-one in DMSO with aqueous hydrochloric acid.

8. A method of isolating a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23; said method comprising the steps of treating the free base of 3-[5-(4-methanesulfonyl-piperazin-1-ylmethyl)-1H-indol-2-yl]-1H-quinolin-2-one in DMSO with aqueous hydrochloric acid and collecting the resulting solid by filtration.

9. The method according to claim 8 that further comprises the step of drying the collected solid under anhydrous conditions.

10. The method according to claim 8 that further comprises the step of drying the collected solid under a nitrogen purge.

11. A method of preparing a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 6.76, 8.12, 10.21, 12.11, 12.88, 13.77, 14.65, 15.01, 15.23, 16.09, 16.36, 16.95, 17.28, 17.65, 18.31, 19.06, 19.66, 20.84, 21.47, 22.21, 23.07, 24.05, 24.32, 25.19, 25.58, 26.00, 26.96, 28.22 and 28.84; said method comprising the step of treating the free base of 3-[5-(4-methanesulfonyl-piperazin-1-ylmethyl)-1H-indol-2-yl]-1H-quinolin-2-one in THF with aqueous hydrochloric acid.

12. The method according to claim 11 that further comprises the step of drying the collected solid under anhydrous conditions.

13. The method according to claim 11 that further comprises the step of drying the collected solid under a nitrogen purge.

14. A method of preparing a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 5.19, 9.54, 10.32, 12.99, 14.79, 15.14, 16.50, 17.10, 17.47, 18.28, 19.12, 19.50, 20.70, 21.00, 21.56, 22.27, 23.24, 24.42, 25.35, 26.06, 26.99, 28.28 and 31.87;
said method comprising the step of recrystallizing a crystalline form of the hydrochloride salt of 3-[5-(4-methanesulfonyl-piperazin-1-ylmethyl)-1H-indol-2-yl]-1H-quinolin-2-one characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23 from a 1:1 acetonitrile/water mixture.

15. A method of preparing a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 5.19, 9.54, 10.32, 12.99, 14.79, 15.14, 16.50, 17.10, 17.47, 18.28, 19.12, 19.50, 20.70, 21.00, 21.56, 22.27, 23.24, 24.42, 25.35, 26.06, 26.99, 28.28 and 31.87;
said method comprising the step of recrystallizing a crystalline form of the hydrochloride salt of 3-[5-(4-methanesulfonyl-piperazin-1-ylmethyl)-1H-indol-2-yl]-1H-quinolin-2-one characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23 from a 1:1 acetone/water mixture.

16. A method of preparing a hydrochloride salt of 3-[5-(4-methanesulfonyl-piperazin-1-ylmethyl)-1H-indol-2-yl]-1H-quinolin-2-one in a crystalline form characterized by an X-ray powder diffraction pattern having diffraction angles of 7.60, 9.350, 11.22, 15.12, 16.01, 16.86, 18.85, 19.46, 20.10, 21.73, 23.07, 23.70, 24.35, 25.99; said method comprising the step of recrystallizing a crystalline form of the hydrochloride salt of 3-[5-(4-methanesulfonyl-piperazin-1-ylmethyl)-1H-indol-2-yl]-1H-quinolin-2-one characterized by an X-ray powder diffraction pattern having diffraction angles of 6.76, 8.09, 9.95, 12.07, 12.85, 13.73, 14.36, 14.85, 15.21, 16.06, 16.34, 16.78, 17.25, 18.29, 18.88, 19.13, 19.72, 20.34, 20.74, 21.55, 22.35, 24.01, 24.24, 25.19, 25.54, 26.86, 28.77 and 30.23 from acetic acid.

17. A pharmaceutical composition that is comprised of a polymorphous form of the hydrochloride salt of 3-[5-(4-methanesulfonyl-piperazin-1-ylmethyl)-1H-indol-2-yl]-1H-quinolin-2-one in accordance with claim 1 and a pharmaceutically acceptable carrier.

18. A pharmaceutical composition that is comprised of a crystalline form of the hydrochloride salt of 3-[5-(4-methanesulfonyl-piperazin-1-ylmethyl)-1H-indol-2-yl]-1H-quinolin-2-one in accordance with claim 3 and a pharmaceutically acceptable carrier.

19. A pharmaceutical composition that is comprised of a crystalline form of the hydrochloride salt of 3-[5-(4-methanesulfonyl-piperazin-1-ylmethyl)-1H-indol-2-yl]-1H-quinolin-2-one in accordance with claim 5 and a pharmaceutically acceptable carrier.

20. A method of treating or preventing cancer in a mammal in need of such treatment which is comprised of administering to said mammal a therapeutically effective amount of the polymorphous form of the hydrochloride salt of 3-[5-(4-methanesulfonyl-piperazin-1-ylmethyl)-1H-indol-2-yl]-1H-quinolin-2-one in accordance with claim 1.

21. A method of treating or preventing cancer in a mammal in need of such treatment which is comprised of administering to said mammal a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

22. A method of treating or preventing cancer in a mammal in need of such treatment which is comprised of administering to said mammal a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 5.

23. A method of treating cancer or preventing cancer in accordance with claim 21 wherein the cancer is selected from cancers of the brain, genitourinary tract, lymphatic system, stomach, larynx and lung.

24. A method of treating or preventing cancer in accordance with claim 23 wherein the cancer is selected from histiocytic lymphoma, lung adenocarcinoma, small cell lung cancers, pancreatic cancer, glioblastomas and breast carcinoma.

25. A method of treating or preventing a disease in which angiogenesis is implicated, which is comprised of administering to a mammal in need of such treatment a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

26. A method in accordance with claim 25 wherein the disease is an ocular disease.

27. A method of treating or preventing retinal vascularization which is comprised of administering to a mammal in need of such treatment a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

28. A method of treating or preventing diabetic retinopathy which is comprised of administering to a mammal in need of such treatment a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

29. A method of treating or preventing age-related macular degeneration which is comprised of administering to a mammal in need of such treatment a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

30. A method of treating or preventing inflammatory diseases which comprises administering to a mammal in need of such treatment a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

31. A method according to claim 30 wherein the inflammatory disease is selected from rheumatoid arthritis, psoriasis, contact dermatitis and delayed hypersensitivity reactions.

32. A method of treating or preventing a tyrosine kinase-dependent disease or condition which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

33. A pharmaceutical composition made by combining the crystalline form of the hydrochloride salt of 3-[5-(4-methanesulfonyl-piperazin-1-ylmethyl)-1H-indol-2-yl]-1H-quinolin-2-one in accordance with claim 3 and a pharmaceutically acceptable carrier.

34. A process for making a pharmaceutical composition which comprises combining the crystalline form of the hydrochloride salt of 3-[5-(4-methanesulfonyl-piperazin-1-ylmethyl)-1H-indol-2-yl]-1H-quinolin-2-one in accordance with claim 3 with a pharmaceutically acceptable carrier.

35. A method of treating or preventing bone associated pathologies selected from osteosarcoma, osteoarthritis, and rickets which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.

36. The composition of claim 18 further comprising a second compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

37. The composition of claim 36, wherein the second compound is another angiogenesis inhibitor selected from the group consisting of a tyrosine kinase inhibitor, an inhibitor of epidermal-derived growth factor, an inhibitor of fibroblast-derived growth factor, an inhibitor of platelet derived growth factor, an MMP inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGF.

38. The composition of claim 36, wherein the second compound is an estrogen receptor modulator selected from tamoxifen and raloxifene.

39. A method of treating cancer which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3 in combination with radiation therapy.

40. A method of treating or preventing cancer which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3 in combination with a compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

41. A method of treating cancer which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3 in combination with radiation therapy and a compound selected from:
1) an estrogen receptor modulator,
2) an androgen receptor modulator,
3) retinoid receptor modulator,
4) a cytotoxic agent,
5) an antiproliferative agent,
6) a prenyl-protein transferase inhibitor,
7) an HMG-CoA reductase inhibitor,
8) an HIV protease inhibitor,
9) a reverse transcriptase inhibitor, and
10) another angiogenesis inhibitor.

42. A method of treating or preventing cancer which comprises administering a therapeutically effective amount of the crystalline form of the hydrochloride salt of 3-[5-(4-methanesulfonyl-piperazin-1-ylmethyl)-H-indol-2-yl]-1H-quinolin-2-one in accordance with claim 3 and paclitaxel or trastuzumab.

43. A method of treating or preventing cancer which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3 and a GPIIb/IIIa antagonist.

44. The method of claim 43 wherein the GPIIb/IIIa antagonist is tirofiban.

45. A method of reducing or preventing tissue damage following a cerebral ischemic event which comprises administering a therapeutically effective amount of the crystalline form of the hydrochloride salt of 3-[5-(4-methanesulfonyl-piperazin-1-ylmethyl)-1H-indol-2-yl]-1H-quinolin-2-one in accordance with claim 3.
